(19) United States
(12) Patent Application Publication
(10) Pub. No.: US 2017/0290892 A1
(43) Pub. Date: Oct. 12, 2017

(54) POLYMERIC ENKEPHALIN PRODRUGS

(71) Applicant: PEG BIOSCIENCES, INC., Monmouth Junstion, NJ (US)

(72) Inventor: Chyi LEE, Princeton Junction, NJ (US)

(73) Assignee: PEG BIOSCIENCES, INC., Monmouth Junction, NJ (US)

(21) Appl. No.: 15/326,063

(22) PCT Filed: Jul. 14, 2015
(86) PCT No.: PCT/US2015/040387

§ 371 Date: Jan. 13, 2017

Related U.S. Application Data
(60) Provisional application No. 62/024,116, filed on Jul. 14, 2014.

(51) Int. Cl. A61K 38/33; C08G 65/332; C08G 65/333; C07K 14/70

(52) U.S. Cl. CPC A61K 38/33; A61K 47/48215; C07K 14/70; C08G 65/332; C08G 65/333; C08L 2203/02; A61K 47/48

(57) ABSTRACT

The unique polymeric OGF and enkephalin peptide conjugates with large size polymer attached at the C-terminus through hydrolysable linkage enhancing therapeutic properties of OGF and enkephalin peptides.

### CROSS-REFERENCE TO RELATED APPLICATIONS

This application claims the benefit of priority of U.S. Provisional Application No. 62/024,116 filed on Jul. 14, 2014, the entire disclosure of which is incorporated herein by reference.

### FIELD OF THE INVENTION

The present invention relates to the polymeric conjugates of enkephalin and related opioid peptides connected to the C-terminus carboxyl group capable of releasing active enkephalin molecules in plasma for achieving increased bioavailability and prolonged circulating life of enkephalin molecules and to methods of making and using such conjugates. More particularly, the invention relates to the releasable polymeric OGF derivatives in which the large size polymer is connected to the OGF's C-terminus carboxyl group through hydrolysable ester linkage without any spacer moiety.

### BACKGROUND OF THE INVENTION

Enkephalins, endorphins and dynorphins are opioid peptides produced by the body. Two forms of enkephalins are met-enkephalin and leu-enkephalin. Met-enkephalin, also referred to as opioid growth factor (OGF), is a naturally occurring, endogenous opioid peptide that has been found to have immuno-regulating and immuno-stimulating effects. OGF has been used to treat several auto immune diseases, including Multiple Sclerosis, Uveitis, Behcet's Syndrome, and Optic Neuritis. OGF has also been reportedly used to treat patients with AIDS. Studies have also demonstrated that OGF significantly raises the levels of natural killer cell, which specialize in killing virus and cancer cells, bacteria, parasites and fungi. In addition, OGF has been found to inhibit angiogenesis that may play an important role in cancer therapy by inhibition of tumor growth and metastasis.

A number of cancers have been shown to have OGF receptors or have been reported to respond to OGF and OGF-boosting mechanisms that include breast cancer, cervical cancer, colorectal cancer, gastric cancer, glioblastoma, head and neck cancer, Kaposi's Sarcoma, lymphocytic leukemia, liver cancer, lymphoma, malignant melanoma, neuroblastoma, ovarian cancer, pancreatic cancer, prostate cancer, renal cell carcinoma, small cell and non-small cell lung cancer, throat cancer, tongue cancer and uterine cancer.

Peptides are in general short-lived species in vivo, having a short circulatory half-life. OGF is a pentapeptide and, like other peptides, has low bioavailability, is rapidly metabolized, and has a very short half-life (minutes). OGF is metabolized by a variety of different enzymes known as enkephalinases. PEG conjugation increases the size and molecular weight of a peptide and protects the peptide from proteolytic degradation resulting in the extension of its half-life in plasma. However, the drawback in pegylation is the reduction or loss of biological activity of the peptide conjugates that are pegylated with either branched or linear PEG compounds. For many therapeutic peptides, a significant loss in biological activity often limits the therapeutic application of pegylated constructs.

There is, therefore, a clearly unmet medical need in the art for effective therapeutic compositions capable of prolonging the half-life of short-lived OGF, enkephalins or peptides without loss of their therapeutic activity.

### SUMMARY OF THE INVENTION

At least one aspect of the present invention provides the novel OGF-polymer or enkephalin-polymer conjugates, which are enkephalins having the C-terminus covalently attached to branched or linear polymers through hydrolysable linkages. The enkephalin-polymer conjugates are the long-acting, controlled continuous release in plasma that provide prolonged half lives and increased bioavailability of the enkephalins as compared to their counterpart unconjugated enkephalins. The enkephalin-polymer conjugate with a large size polymer slowly releases enkephalin in plasma and can be considered to be a prodrug of enkephalin.

At least one aspect of the present invention provides enkephalin-polymer conjugates comprising the formula:

[Enkephalin-C(O)—X]ₙ—P Formula I

wherein P is polymer, polymer-lipid or polymer derivatives attached to the C-terminus carboxyl group without spacer between terminal amino acid and polymer; wherein X is an atom or a chemical moiety selected from the group consisting of O, S, imidazo, and phosphate; wherein C(O)—X is the hydrolysable linkage comprising the functional group selected from carboxylic ester, thioester, acylimidazo, and phosphonic ester; wherein n>1, depending on the availability of attachment sites on polymer P; wherein enkephalins attached to polymer include, but not limited to, OGF (met-enkephalin), leu-enkephalin, endorphins, dynorphins, proenkephalins and synthetic enkephalin analogues.

Where P is a PEG polymer and X is oxygen the conjugates have the formula:

[Enkephalin-C(O)—O]ₙ—PEG Formula II

wherein the PEG polymer is directly connected to the C-terminus carboxyl group through ester linkage without any spacer between the C-terminus and PEG polymer; wherein n≧1, depending on the number of attachment sites on the PEG (n=1 or 2 for linear PEGs, n>1 for multi-arm PEGs); wherein PEG is selected from the group of mPEG (methoxy polyethylene glycol), linear PEG, branched PEG, multiple-arm PEG, PEG-lipid, copolymers, block copolymers, terpolymers, and PEG polymer derivatives capable of forming hydrogels, liposomes or nanoparticles.

At least one aspect of the present invention is directed to OGF-PEG (met-enkephalin-PEG) ester conjugates wherein the enkephalin is OGF and the conjugate is represented by the formula:

[OGF—C(O)—O]ₙ—PEG

Or

[H₂N-Tyr-Gly-Gly-Phe-Met-C(O)—O]ₙ—PEG Formula III

At least one aspect of the present invention provides the novel peptide-PEG conjugates comprising the formula:

[Peptide-C(O)—O]ₙ—PEG Formula IV

wherein the PEG polymer directly connected to the peptide's C-terminus carboxyl group through ester linkage without spacer between the peptide's amino acid and PEG polymer; wherein n≧1; wherein PEG selected from the group of mPEG (methoxy polyethylene glycol), linear PEG, branched PEG, multiple-arm PEG, PEG-lipid, copolymers, block copolymers, terpolymers, and PEG polymer derivatives capable of forming hydrogel, liposome and nanoparticle.

One of the advantages of coupling of hydroxyl group of PEG polymer directly to the C-terminus of OGF, enkephalin or peptide without any spacer between PEG and the carboxyl groups is the avoidance of toxicity and immunogenicity issues caused by the linking spacer molecules.

Another advantage of the invention is that the releasable OGF-PEG, enkephalin-PEG or peptide-PEG conjugates not only release their parent molecules (OGF, enkephalins or peptide, respectively) in plasma to increase their bioavailability, but also the conjugates provide prolonged circulation time to enhance their therapeutic efficacy.

Attaching a large size mPEG-OH (e.g. 20,000 Dalton) directly to the OGF C-terminus to form the OGF-C(O)O—PEG ester conjugates is difficult because of the inherent steric hindrance from both the bulky size PEG and the OGF pentapeptide, and also the relatively nonreactive hydroxyl group —OH of PEG-OH. In practice this makes the directly conjugated peptide-C(O)—O—PEG constructs unavailable.

Our solution to this problem is to use a pre-synthesized H₂N-methionine-C(O)—O—PEG ester derivative to replace the OGF's C-terminal methionine and then couple to the carboxyl group of the tetrapeptide tyr-gly-gly-phe-CO₂H via the formation of amide bond to yield the releasable OGF-PEG conjugate (H₂N-Tyr-Gly-Gly-Phe-Met-C(O)—O—PEG). In a further embodiment, a pre-synthesized H₂N-methionine-C(0)-O—PEG ester or other amino acid-PEG ester derivative can be coupled with the pentapeptide OGF itself to form a conjugate containing an additional amino acid.

The strategy of using a pre-synthesized H₂N-amino acid-C(O)—O—PEG ester derivative to replace the C-terminal amino acid of a peptide is critical for the synthesis of the hydrolysable peptide-PEG ester conjugate (Formula II, III and IV).

At least one aspect of the invention provides the hydrolysable H₂N-amino acid-C(O)—O-mPEG derivatives (AA-PEG, Formula V) to replace the corresponding C-terminal amino acids for the synthesis of the releasable enkephalin-PEG, OGF-PEG and peptide-PEG ester conjugates in formula II, III and IV, respectively. For synthesis of OGF-PEG conjugate, AA-PEG is H₂N-methionine-C(O)—O-mPEG. For synthesis of leu-enkephalin-PEG, AA-PEG is H₂N-leucine-C(O)—O-mPEG.

AA-PEG or H₂N-amino acid-C(O)—O—PEG Formula V

wherein the AA is the C-terminal amino acid of a peptide includes, but not limited to, methionine, glycine, alanine, phenylalanine, leucine, isoleucine, serine, threonine, glutamine, asparagine, aspartic acid, glutamic acid, histidine, cysteine, tyrosine, lysine, arginine, proline, tryptophan, valine and homo-amino acids.

The hydrolysable AA-PEG reagents having amino acids with ester linkage are useful for preparing a releasable PEG-peptide conjugate. The releasable AA-PEG derivatives can be covalently coupled to a peptides' C-terminus or carboxyl groups to provide the releasable peptide-PEG conjugates (Formula VI).

[Peptide-C(O)—NH-amino acid-C(O)—O]ₙ—PEG Formula VI

The H₂N-amino acid-C(O)—O—PEG derivatives are preferably attached to the peptide's C-terminus carboxyl group. For example, the structure of OGF—X-AA-PEG is represented as follows:

H₂N-Tyr-Gly-Gly-Phe-Met-X-AA-PEG

Or

[H₂N-Tyr-Gly-Gly-Phe-Met-X—C(O)—NH-AA-PEG

wherein X is a peptide or an amino acid. OGF—X peptide is a synthetic analogue of OGF.

The releasable AA-PEG reagent is also useful for creating a releasable biologic-PEG conjugate by attaching it to the biologics or small molecules at their electrophilic groups selected from the group consisting of N-hydroxysuccinimide ester, p-nitophenyl ester, N-succinimidyl carbonate, p-nitrophenyl carbonate, carboxyl, carbonyl and aldehyde.

### DETAILED DESCRIPTION OF PREFERRED EMBODIMENTS

Enkephalins, endorphins, and dynorphins are three well-characterized families of opioid peptides produced by the body. Two forms of enkephalin are the products of the proenkephalin gene. They are Met-enkephalin(OGF, Tyr-Gly-Gly-Phe-Met) and leu-enkephalin (Tyr-Gly-Gly-Phe-Leu).

OGF has many biologic effects, such as analgesia, inhibition of angiogenesis, immune-stimulating and immuno-regulating properties, etc. OGF inhibits tumor growth and increases the number and functions of T cells, natural killer (NK) cells, and NK-T cells to destroy virally infected cells and cancer.

OGF or enkephalin peptide has low bioavailability, is rapidly metabolized, and has a very short half-life (minutes). Prolonged maintenance of therapeutically OGF or enkephalin peptide drugs in circulation is a desirable feature of primary clinical importance. Pegylation has been used to enhance proteins and peptides stability and circulation, while reducing proteolysis, immunogenicity, and clearance. However, the drawback is the loss of biological activity due to the steric hindrance created by the large PEG polymer size and/or the attachment of PEG at peptide's active binding sites. For PEG-conjugated [D-Pen2, D-Pen5]-enkephalin (DPDPE), modification at N-terminus with 2k PEG resulted in a significant loss of binding affinity for the δ-opioid receptor was described by Witt, et al. in J Pharmacol Exp Ther 298: 848-856.

The present invention relates to enkephalin-polymeric conjugates having a polymer covalently bonded to enkephalin at the C-terminus through hydrolysable linkages and to methods of making and using such conjugates. The formula is represented as follows:

[Enkephalin-C(O)—X]ₙ—P Formula I

wherein enkephalins attached to polymer include, but not limited to, met-enkephalin (OGF), leu-enkephalin, endorphins, dynorphins, proenkephalins and synthetic enkephalin analogues; wherein X is an atom or a chemical moiety selected from the group consisting of O, S, imidazo, and phosphate; wherein C(O)X is the functional group selected from ester, thioester, acylimidazo, and phosphonic ester; wherein n≧1; wherein P is a polymer, polymer-lipid or polymer derivative.

Enkephalins may also include other neuropeptides, such as met-enkephalin-Arg-Phe, met-enkephalin-Arg-Gly-Leu, adrenorphin, amidorphin, BAM-18, BAM-20P, BAM-22P, peptide B, peptide E, peptide F and a-neoendorphin. proenkephalins include proenkephalin A (known as proenkephalin or PENK) and proenkephalin B (prodynorphin).

The polymers used for enkephalin-polymer conjugates are preferably water-soluble. A non-limiting list of such polymers include polyalkylene oxide homopolymers such as polyethylene glycol (PEG) or polypropylene glycol, poly(vinyl alcohol), poly(oxyethylated glycerol), poly(oxyethylated sorbitol), poly(oxyethylated glucose), poly(oxazoline), poly(acryloylmorpholine), poly(vinylpyrrolidone), polyoxyethylenated polyol, branched polymer, copolymer, block copolymer, terpolymer, and mixtures thereof. The polyalkylene oxides containing alkyl terminals, such as monomethoxy polyethylene glycols (mPEG) are also included. The polymer includes the polymer derivatives capable of forming hydrogel, liposome and nanoparticle.

In one aspect of the present invention is the instantly disclosed enkephalin-PEG ester conjugates (Formula II) capable of releasing active enkephalins in plasma and methods of making and using such conjugates. The enkephalin-PEG conjugate comprises a PEG polymer connected directly to enkephalins at C-terminus through hydrolysable ester linkages without any spacer moiety. The enkephalin-PEG conjugate is represented by the formula:

[Enkephalin-C(O)—O]ₙ—PEG Formula II

wherein the enkephalin-PEG conjugate comprising a PEG polymer connected directly to enkephalins at C-terminus through hydrolysable linkages; wherein enkephalins attached to PEG polymer include, but not limited to, met-enkephalin (OGF), leu-enkephalin, endorphins, dynorphins, proenkephalins and synthetic enkephalin analogues; wherein —C(O)—O is the hydrolysable carboxylic ester linkage connected directly between enkephalin's C-terminus and PEG; wherein PEG is selected from the group of mPEG (monomethoxy polyethylene glycol), linear PEG, branched PEG, multiple-arm PEG, PEG-lipid, copolymers, block copolymers, terpolymers, and PEG polymer derivatives capable of forming hydrogel, liposome and nanoparticle.

Preferably, the enkephalin is OGF (met-enkephalin) or leu-enkephalin. The conjugate is represented by the formula:

[OGF—C(O)—O]ₙ—PEG

or

[Leu-enkephalin-C(O)—O]ₙ—PEG Formula III

wherein n=1; wherein mPEG is methoxy polyethylene glycol; OGF-PEG or leu-enkephalin-PEG with linear PEG polymer is represented by the formula:

OGF—C(O)—O-mPEG

(H₂N-Tyr-Gly-Gly-Phe-Met-C(O)—O-mPEG)

Or

Leu-enkephalin-C(O)—O-mPEG

(H₂N-Tyr-Gly-Gly-Phe-leu-C(O)—O-mPEG)

The PEG or mPEG has a molecular weight ranging from about 200 to about 40,000 Daltons, preferably from about 1000 to about 40,000 Daltons, more preferably from about 5,000 to about 40,000 Daltons, and still more preferably from about 20,000 to about 40,000 Daltons.

When n=2, example of preferred OGF-PEG or leu-enkephalin-PEG conjugate for linear PEG polymer is represented by the formula:

OGF—C(O)—O—PEG-O—(O)C—OGF

(H₂N-Tyr-Gly-Gly-Phe-Met-C(O)—O—PEG-O—(O)C-Met-Phe-Gly-Gly-Tyr-NH₂)

Or

Leu-enkephalin-C(O)—O—PEG-O—(O)C-enkephalin-Leu

(H₂N-Tyr-Gly-Gly-Phe-Leu-C(O)—O—PEG-O—(O)C-Leu-Phe-Gly-Gly-Tyr-NH₂)

[OGF-C(O)—O]ₙ—PEG may contain multiple OGF molecules when PEG is multi-arm PEG. For example, multi-arm PEG can be 4-arm PEG or 8-arm PEG, etc. The combined molecular weight of the multi-arm PEG polymer is in the range about 2,000 to about 40,000 Daltons, preferably about 20,000 to about 40,000 Daltons.

Coupling of high molecular weight of polyethylene glycol (PEG) to enkephalin peptides with hydrolysable linkage are useful in the delivery active enkephalins in vivo. One of major advantages disclosed in the instant enkephalin-PEG conjugates with hydrolysable ester linkage at C-terminus is the ability of the conjugates can prolong enkephalin peptides' half-life and increase their bioavailability in plasma. The renal filtration decreases at higher molecular weight of PEGs above 20 kDa and thus, in favor of a large size PEG-enkephalin conjugate providing slowly released enkephalin circulation in vivo.

Another advantage of coupling of hydroxyl group of PEG directly to enkephalin or peptide's C-terminus without any spacer between PEG and peptide may avoid the toxicity and immunogenicity problems caused by a spacer moiety.

The invention provides a method for the preparation of peptide-PEG ester linkage conjugate possessing no linking spacer moiety between peptide and PEG.

Synthesis of OGF—PEG ester conjugate is problematic by directly coupling of hydroxyl group of large size PEG molecule to the C-terminus carboxyl group of OGF because of the inherent steric hindrance of the large size PEG and the OGF pentapeptide molecules and the relative nonreactive hydroxyl group OH of PEG.

A strategy of using the pre-synthesized C-terminal amino acid methionine-PEG ester derivative (H₂N-Met-C(O)—O-mPEG) is the key for synthesis of OGF-mPEG ester conjugate. The pre-synthesized H₂N-Met-C(O)—O-mPEG containing free a-amino group can therefore efficiently couple to the tetrapeptide (Boc-HN-Tyr-Gly-Gly-Phe-CO₂H) via the formation of amide bond between the large size H₂N-Met-C(O)—O—PEG and tetrapeptide-CO₂H molecules.

Therefore, OGF-mPEG ester (met-enkephalin-mPEG ester) conjugate is synthesized by coupling Boc-HN-Tyr-Gly-Gly-Phe and H₂N-Met-C(O)—O-mPEG and follows by deblocking Boc group.

The synthetic scheme is shown as follows:

Boc-HN-Met-C(O)OH+mPEG-OH Boc-HN-Met-C(O)—O-mPEG

Boc-HN-Met-C(O)—O-mPEG+TFA H₂N-Met-C(O)—O-mPEG

Boc-HN-Tyr-Gly-Gly-Phe+H₂N-Met-C(O)—O-mPEG

→Boc-H₂N-Tyr-Gly-Gly-Phe-Met-C(O)—O-mPEG

Boc-H₂N-Tyr-Gly-Gly-Phe-Met-C(O)—O-mPEG+TFA H₂N-Tyr-Gly-Gly-Phe-Met-C(O)—O-mPEG

Th strategy of using the pre-synthesized C-terminal amino acid-PEG ester (AA-PEG) to replace a peptide's C-terminal amino acid are beneficial for biologically active peptide to provide a releasable peptide-PEG conjugate without a spacer moiety between the peptide and PEG polymer. A spacer moiety on protein and peptide may cause immunogenicity and toxicity side effects. In addition, the parent peptide amino acids sequence is intact in the synthesized releasable peptide-PEG conjugate by using the methodology of the pre-synthesized AA-PEG.

The unique releasable peptide-PEG conjugates comprising the formula:

[Peptide-C(O)—O]ₙ—PEG Formula IV

wherein n≧1; wherein said PEG polymer directly connected to the peptide's C-terminus carboxyl group through ester linkage without spacer between the peptide's amino acid and

PEG polymer; wherein said PEG selected from the group of mPEG (methoxy polyethylene glycol), linear PEG, branched PEG, multiple-arm PEG, PEG-lipid, copolymers, block copolymers, terpolymers, and PEG polymer derivatives capable of forming hydrogel, liposome and nanoparticle.

The [peptide-C(O)—O]ₙ—PEG conjugate provides a controlled, continuous releasing system of active peptide in plasma. One of the advantages of the invention is that the releasable peptide-PEG conjugates not only increase the bioavalability by releasing the parent peptide molecules slowly in plasma, but also the conjugates provide prolonged plasma circulation time to increase half livesof the peptide molecules. Another advantage of the invention is the unique releasable peptide-PEG conjugate having PEG polymer directly connected to the peptide's C-terminus carboxyl group without any spacer moiety between PEG and the carboxyl group that can avoid the toxicity and immunogenicity side effects caused by the linking spacer molecules.

However, synthesis of desired peptide-C-terminus-PEG ester conjugate by attaching a large size mPEG-OH directly to a peptide's (e.g. pentapeptide) C-terminus is problematic because of the inherent steric hindrance from both the bulky size PEG and peptide plus the relatively nonreactive hydroxyl group —OH of PEG-OH.

A novel approach to the problem is to use a pre-synthesized H₂N-amino acid-C(O)—O—PEG ester derivative (AA-PEG) containing free a-amino group that can efficiently couple to peptide to generate a covalent amide bond between large size H₂N-amino acid-C(O)—O—PEG and peptide-CO₂H molecules. This methodology provides an efficient way for synthesizing peptide-PEG conjugate that can release an intact peptide molecule in plasma.

The pre-synthesized H₂N-amino acid-C(O)—O—PEG ester derivative (AA-PEG) replacing the C-terminal amino acid of a peptide is represented by formula:

H₂N-Amino acid-C(O)—O—(CH₂CH₂O)ₙCH₂CH₂—OCH₃ (AA-mPEG)

Or

H₂N-Amino acid-C(O)—O—(CH₂CH₂O)ₙ—O—(O)C-Amino Acid-NH₂ (AA-PEG-AA) Formula V

wherein n is about 4 to about 1000, preferably about 10 to about 1000, preferably about 20 to about 1000, more preferably about 50 to about 1000, still more preferably about 100 to about 1000, still more preferably about 250 to about 1000, most preferably about 500 to about 1000. In a further embodiment, a pre-synthesized H₂N-amino acid-C(O)—O—PEG ester derivative (AA-PEG) can be reacted with the pentapeptide OGF itself to form a conjugate containing an additional amino acid.

When the amino acid (AA) is the C-terminal amino acid of a peptide, AA includes but is not limited to, glycine, alanine, phenylalanine, leucine, isoleucine, serine, threonine, glutamine, asparagine, aspartic acid, glutamic acid, histidine, cysteine, tyrosine, lysine, arginine, proline, tryptophan, valine and homo-amino acids.

The strategy of using a pre-synthesized H₂N-amino acid-C(O)—O—PEG ester derivative to replace the C-terminal amino acid of a peptide is critical for the synthesis of a releasable peptide-PEG ester conjugate, such as enkephalin-PEG (formula II), OGF-PEG and leu-enkephalin-PEG (formula III), and peptide-PEG (formula IV). This strategy can be applied for various PEG size polymers, especially for PEG polymer size equal or larger than 5,000 Daltons.

The releasable H₂N-amino acid-C(O)—O—PEG derivatives can also be used to couple with a peptide's C-terminus or carboxyl groups to generate a peptide-PEG conjugate with hydrolysable ester linkage. The H₂N-amino acid-C(O)—O—PEG-PEG derivative is preferably attached to the peptide's C-terminus carboxyl group.

The conjugate formula is represented as follows:

[Peptide-NH-amino acid-C(O)—O]ₙ—PEG Formula VI

The synthetic scheme is represented as follows:

Peptide-CO₂H+H₂N-amino acid-C(O)—O—PEG→Peptide-C(O)—NH-amino acid-C(O)—O—PEG

wherein the desired amino acid, includes but not limited to, glycine, alanine, phenylalanine, leucine, isoleucine, serine, threonine, glutamine, asparagine, aspartic acid, glutamic acid, histidine, cysteine, tyrosine, lysine, arginine, proline, tryptophan, valine and homo-amino acids.

For example, if peptide is OGF—X derivative, the releasable OGF conjugate is represented by formula:

H₂N-Tyr-Gly-Gly-Phe-Met-X-amino acid-C(O)—O—PEG Formula VII

wherein X is a peptide or an amino acid. OGF-X peptide is a synthetic analogue or derivative of OGF.

The hydrolysable amino acid-C(O)—O—PEG is also useful for creating a releasable PEG-biologic with ester linkage by attaching amino of the amino acid-C(O)—O—PEG reagent to biologics at their electrophilic groups selected from the group consisting of N-hydroxysuccinimide ester, p-nitophenyl ester, N-succinimidyl carbonate, p-nitrophenyl carbonate, carboxyl, carbonyl and aldehyde.

The releasable peptide-PEG conjugates as described in Formula IV or VI can increase the retention of peptides in the circulation by protecting against enzymatic digestion, reducing excretion by the kidneys and slowly releasing the active peptides.

The releasable OGF- PEG peptide conjugates as shown in Formula III or VIII are capable of providing three major effects; a decrease in the rate of kidney clearance, an increase in protection from proteolytic degradation and a slow release of active OGF peptide. The unique releasable OGF- PEG peptide conjugates provide prolonged circulation and improved bioavailability of OGF for many potential treatment of cancer, autoimmune and infectious diseases.

OGF has been found to inhibit angiogenesis and significantly raises natural killer cell levels to destroy virally infected cells and cancer. OGF has been used for the treatment of advanced pancreatic cancer patients. The OGF- PEG peptide conjugates of the invention have potential to be used for the treatment of many cancer diseases, including but not limited to, Pnacreatic cancer, Lung cancer, Breast Cancer, Cervical Cancer, Colon and Rectal Cancer, Gastric Cancer, Glioblastoma, Head and Neck, Liver Cancer, Neuroblastoma, Ovarian Cancer, Prostate cancer and Multiple Myeloma. The OGF- PEG peptide conjugates can also be given in combination with other types of cancer treatment, such as chemotherapy, targeted therapy, immunotherapy, radiation therapy, photodynamic therapy, etc.

OGF has immune-regulating and immune-stimulating effects and has been used for the treatment of several autoimmune diseases, including multiple sclerosis, Uveitis, Behcet's Syndrome, and Optic Neuritis. It has also been reported that reduced level in Parkinson, Crohn, inflammatory bowel diseases, etc. The presently disclosed OGF-PEG peptide conjugates of the invention have potential to be used for the treatment of many autoimmune diseases, including but not limited to, multiple sclerosis, Uveitis, Behcet's Syndrome, Optic Neuritis, Parkinson disease, Crohn disease, ulcerative colitis and inflammatory bowel disease. The PEG-OGF peptide conjugates can also be given in combination with other drugs for the treatment of autoimmune diseases.

OGF modulates and activates dopamine neurons in ventral tegmental area. OGF-PEG may stop the gradual, progressive death of neurons or dopamine neurons, preventing a loss of function of the nervous system. The presently disclosed OGF-PEG peptide conjugates of the invention may have therapeutic potential for many neurologucal and neurodegenerative diseases or conditions, including but not limited to, Alzheimer's disease, Parkinson's disease, Lewy body diseases, Huntington's disease, Lou Gehrig's disease, Schizophrenia, neuropathic pain, seizure, autism, drug addiction (heroin, cocaine, marijuana, etc.) and depression. The OGF-PEG peptide conjugates can also be given in combination with other drugs for the treatment of neurological and neurodegenerative diseases and conditions.

OGF raises natural killer cell levels to destroy virally infected cells, bacteria, parasites, and fungi. The OGF-PEG peptide conjugates have the potential to be an effective medicine for the treatment of infectious diseases, including but not limited to,

HIV/AIDS, viral hemorrhagic fevers, Dengue fever and malaria. The OGF-PEG peptide conjugates can also be given in combination with other antiviral drugs for the treatment of viral diseases.

The OGF- PEG conjugates of the invention are the enzyme controlled, continuous release systems of OGF that provide sustained biological activity of OGF in plasma. The OGF- PEG conjugates provide prolonged drug circulation and improved bioavailability for enhancing therapeutic efficacy.

The invention provides the methodology of synthesis of releasable peptides' C-terminus-PEG conjugates through ester linkage and amino acid-PEG carboxyl ester derivatives for peptide or biomolecule conjugation. The synthetic methodology includes, but not limited to, the protection, de-protection, activation and insertion methods and procedures for the synthesis of releasable peptides' C-terminus-PEG conjugates.

The invention also provides for a method of preparing releasable peptide-PEG conjugates at C-terminus without a spacer between peptide and PEG polymers.

The invention also provides for a method of preparing releasable enkephalin-PEG conjugates at C-terminus without a spacer between enkephalin and PEG polymers.

### EXAMPLES

The following non-limiting examples illustrate certain aspects of the invention.

### Example 1

### Synthesis of 20K Boc-Met-C(O)O-mPEG (20K Boc-Met-mPEG ester)

To a solution of 20 kDa mPEG (4g, 0.2 mmol) and Boc-L-methionine (252mg, 1.0 mmol) in dry DCM cooled in an ice-water bath was added dicyclohexylcarbodiimide (783 mg, 3.8 mmol), and the mixture was stirred under nitrogen and allowed to warm to room temperature overnight. The N,N′-dicyclohexylurea was removed from the reaction mixture by filtration. The filtrate was concentrated in vacuo and the white solid product precipitated with ethyl ether. The white solid product was collected and washed with ethyl ether to give 20K Boc-Met-C(O)O-mPEG (3.4 g).

### Example 2

### Synthesis of 20K Met-C(O)O-mPEG (20K Met-mPEG ester)

To a solution of 20K Boc-Met-C(0)O-mPEG (1.3 g, 0.065 mmol) and thioanisole (0.8 mL) in dry DCM (11 mL) was added trifluoroacetic acid (5.7 ml) and the solution was stirred at room temperature for 30 minutes. The solution was concentrated in vacuo and the product was precipitated with ethyl ether, filtered, collected, and washed with ethyl ether to yield 20K Met-C(O)O-mPEG (1.2 g).

### Example 3

### Synthesis of 20K Boc-Tyr-Gly-Gly--Phe-Met-C(O)O-mPEG (20K Boc-Met-enkephalin-mPEG ester)

A solution of Boc-Tyr-gly-gly-phe (65 mg, 0.12 mmol), 20K Met-C(O)O-mPEG (1.2 g, 0.06 mmol), N-hydroxysuccinimide (7 mg, 0.06 mmol) and DMAP (66 mg, 0.54 mmol) in a mixture of DCM/DMF (14 mL/7 mL) was cooled to 0° C., followed by the addition of EDC (107 mg, 0.55 mmol). The mixture was allowed to warm to room temperature and stirred overnight. The solution was concentrated in vacuo and the product precipitated with ethyl ether, filtered, collected, and washed with ethyl ether to yield 20K Boc-Tyr-Gly-Gly-Phe-Met-C(O)OPEG (1.2 g).

### Example 4

### 20K Tyr-Gly-Gly-Phe-Met-C(O)O-mPEG (20K OGF-mPEG ester or 20K Met-enkephalin-mPEG ester)

To a solution of 20K Boc-Tyr-Gly-Gly-Phe-Met-C(O)O-mPEG (1.2 g), m-cresol (0.8 mL) and thioanisole (0.8 mL) in DCM (10 mL) was added trifluoroacetic acid (TFA, 5.7 mL) and the solution was stirred for 30 minutes at room temperature. The solvent was partially removed under reduced pressure and the product precipitated with ethyl ether, filtered, and the solid was dissolved in DCM and washed with 0.01N HCl. The organic layer was dried (anhydrous magnesium sulfate), filtered, and the solution was concentrated in vacuo and the product precipitated with ethyl ether to yield 20K Tyr-Gly-Gly-L-Phe-Met-C(O)—O-mPEG (1.0 g).

### Example 5

### 20K OGF-PEG Conjugate with Hydrolysable Ester Linkage in Human Plasma

Experiments for degradation of the 20K Tyr-gly-gly-phe-met-C(O)—O-mPEG conjugate (20K OGF-C(O)—O-mPEG or 20K OGF—PEG ester) and the release of OGF were conducted in human plasma at 37 ° C. The OGF—PEG conjugate containing hydrolysable ester linkages was incubated in human plasma at 37° C. for various periods of time, ranging from 0.5 to 24 hours. An aliquot plasma sample was withdrawn and treated with acetonitrile or acetonitrile/methanol organic solvent, vortexed, centrifuged, and concentrated. The solution residue was then analyzed by reversed-phase HPLC chromatography or size exclusion chromatography to check the amount of OGF-PEG and the released amount of OGF at various time points. The plasma T1/2 for 20K OGF-C(O)—O-mPEG was about 5 hours.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and scope of the invention, and all such variations are intended to be included within the scope of the following claims.

1-17. (canceled)

18. An enkephalin-polymer conjugate of the formula:
[Enkephalin-C(O)—X]n-P
wherein P is polymer, polymer-lipid or polymer derivative attached to the C terminus carboxyl group without spacer between terminal amino acid and polymer;
wherein X is an atom or a chemical moiety selected from the group consisting of O, S, imidazo, and phosphate;
wherein C(O)—X is the hydrolysable linkage comprising the functional group selected from carboxylic ester, thioester, acylimidazo, and phosphonic ester;
wherein n≧1;
wherein enkephalins attached to polymer is selected from the group consisting of endogenous enkephalins (including OGF (met-enkephalin) and leu-enkephalin), opioid peptides, proenkephalins, endorphins, dynorphins, proenkephalins and synthetic enkephalin analogues.

19. The enkephalin-polymer conjugate of claim 18, wherein P is PEG and X is O, providing the formula:
[Enkephalin-C(O)—O]ₙ—PEG
wherein PEG is selected from the group consisting of mPEG (monomethoxy polyethylene glycol), linear PEG, branched PEG, multiple-arm PEG, PEG-lipid, PEG copolymers, PEG block copolymers, PEG terpolymers, and PEG polymer derivatives capable of forming hydrogels, liposomes or nanoparticles.

20. The enkephalin-polymer conjugate of claim 19, wherein said enkephalin is OGF, such that the C-terminus methionine carboxylic ester linkage is connected directly to a PEG polymer.

21. The OGF-PEG conjugate of claim 20, wherein said PEG is methoxypolyethylene glycol (m PEG); wherein n=1.

22. The OGF-PEG conjugate of claim 20, wherein n=2.

23. A method of preparing the OGF—PEG conjugates of claim 20, comprising:
reacting the tetrapeptide Tyr-Gly-Gly-Phe with pre-synthesized Met-C(O)O- mPEG.

24. The conjugate of claim 19, wherein the enkephalin is an OGF—X derivative, wherein X is a peptide or an amino acid.

25. The enkephalin-polymer conjugate of claim 19, wherein said enkephalin is leu- enkephalin, such that the C-terminus leucine carboxylic ester linkage is connected directly to a PEG polymer.

26. The leu-enkephalin-PEG conjugate of claim 25, wherein said PEG is methoxypolyethylene glycol (m PEG); wherein n=1.

27. The leu-enkephalin-PEG conjugate of claim 22, wherein n=2.

28. A peptide-PEG conjugate having the formula:
[Peptide-C(O)—O]ₙ—PEG
wherein n≧1 ;
wherein —C(O)—O is the peptide C-terminus carboxylic ester linkage connected directly to a PEG polymer; and
wherein PEG is selected from the group consisting of mPEG (monomethoxy polyethylene glycol), linear PEG, branched PEG, multiple-arm PEG, PEG-lipid, PEG copolymers, PEG block copolymers, PEG terpolymers, and PEG polymer derivatives capable of forming hydrogels, liposomes or nanoparticles.

29. A method of preparing the peptide-PEG conjugate having a hydrolysable ester linkage of claim 28, comprising:
replacing a peptide's C-terminal amino acid with a pre-synthesized AA-C(O)O- mPEG.

30. A pre-synthesized C-terminal amino acid-PEG ester derivative having the formula:
Pₘ-AA-m PEG
or
(Pₘ-AA)_{q}-PEG
wherein PEG is linear PEG, branched PEG or multiple-arm PEG;
wherein mPEG is monomethoxy polyethylene glycol and having structure —O—(CH₂CH₂O)ₙ—CH₃;
wherein n is about 4 to about 1000;
wherein m is 0 or 1;
wherein q≧1;
wherein P-AA comprises a polypeptide; and
wherein AA is the C-terminal amino acid of the peptide, or a desired amino acid, and said AA is selected from the group consisting of glycine, alanine, phenylalanine, leucine, isoleucine, serine, threonine, glutamine, asparagine, aspartic acid, glutamic acid, histidine, cysteine, tyrosine, lysine, arginine, proline, tryptophan, valine and other homo-amino acids.

31. A peptide-PEG conjugate with a hydrolysable ester linkage according to claim 30 and having the formula:
[Peptide-C(O)—NH-AA-C(O)—O]_{q}—PEG.

32. A method of preparing the peptide-PEG conjugate having a hydrolysable ester linkage of claim 30, comprising:
reacting a carboxyl group of a peptide with a pre-synthesized AA-C(O)O-mPEG.

33. The C-terminal amino acid-PEG ester derivative of claim 30, wherein AA is methionine, having the structure:
H₂N-methionine-C(O)—O—(CH₂CH₂O)ₙCH₂CH₂—OCH3.

34. A method of forming a releasable biologic which is B or B-AA, comprising reacting AA-mPEG or AA-PEG-AA with B having one or more of the following reactive groups: N-hydroxysuccinimide ester, p-nitophenyl ester, N-succinimidyl carbonate, p-nitrophenyl carbonate, carboxyl, carbonyl or aldehyde.

35. A method of treating a neurological or neurodegenerative disease or disorder, comprising administering to a subject in need thereof an effective amount of the conjugate of claim 19, wherein neurological or neurodegenerative disease is Alzheimer's disease, Parkinson's disease, Lewy body diseases, Huntington's disease, Lou Gehrig's disease, Schizophrenia, neuropathic pain, seizure, autism, drug addiction or depression.

36. A method of treating a cancer disease or disorder, comprising administering to a subject in need thereof an effective amount of the conjugate of claim 19; wherein cancer disease or disorder includes is pancreatic cancer, lung cancer, breast cancer, cervical cancer, colon and rectal cancer, gastric cancer, glioblastoma, head and neck, liver cancer, neuroblastoma, ovarian cancer, prostate cancer or multiple myeloma.

37. A method of treating an autoimmune disease or disorder, comprising administering to a subject in need thereof an effective amount of the conjugate of claim 19; wherein autoimmune disease is multiple sclerosis, Uveitis, Behcet's Syndrome, Optic Neuritis, Parkinson disease, Crohn disease, ulcerative colitis or inflammatory bowel disease.

38. A method of treating a viral infection, bacteria, parasites, or fungi disease or disorder, comprising administering to a subject in need thereof an effective amount of the conjugate of claim 19.

39. The enkephalin-polymer conjugate in claim 19, wherein the plasma cleavage provides controlled release of parent enkephalin molecules for delivering active enkephalins in vivo and provides prolonged enkephalin drugs circulation as well as improved bioavailability for the treatment of enkephalin-deficiency or enkephalin-related diseases or disorders.


SEQUENCE LISTING (3 sequences)

SEQ ID NO: 1 - protein, 5 aa, Homo sapiens
YGGFM

SEQ ID NO: 2 - protein, 4 aa, Homo sapiens
YGGF

SEQ ID NO: 3 - protein, 5 aa, Homo sapiens
YGGFL